(19) United States
(12) Patent Application Publication
(10) Pub. No.: US 2020/0085335 A1
(43) Pub. Date: Mar. 19, 2020

(54) SUCTION CUP EKG ELECTRODE

(71) Applicant: Anand Morankar, New York, NY (US)

(72) Inventor: Anand Morankar, New York, NY (US)

(21) Appl. No.: 16/469,719

(22) PCT Filed: Dec. 14, 2017
(86) PCT No.: PCT/US2017/066273

§ 371 Date: Jun. 14, 2019

Related U.S. Application Data
(60) Provisional application No. 62/433,851, filed on Dec. 14, 2016.

(51) Int. Cl. A61B 5/0408; A61B 5/00

(52) U.S. Cl. CPC A61B 5/04082; A61B 2562/0209; A61B 5/6834

(57) ABSTRACT

A suction cup electrode and methods thereof configured to be used with an electrocardiogram (EKG or ECG). The suction cup electrode is reusable and creates an effective comfortable suction to most skin surfaces of most patients and is able to be applied to and removed from patients with minimal force and discomfort for patients.

### CROSS REFERENCE TO RELATED APPLICATION

This application claims priority to U.S. 62/433,851 entitled “Suction Cup EKG electrode” filed on Dec. 14, 2016. This application is incorporated by reference in its entirety as if fully set forth herein.

### FIELD OF THE INVENTION

A suction cup electrode and methods thereof configured to be used with an electrocardiogram (EKG or ECG). The EKG suction cup electrode is configured to create an effective suction to the skin of a patient while being comfortable and being able to be applied to and removed from a patient with minimal force, while providing low levels of discomfort or minimal to zero discomfort. The EKG suction cup electrode is configured to be reusable on multiple patients with cleaning and/or sterilization of the suction cup electrode between uses.

### BACKGROUND OF THE INVENTION

An electrocardiogram (EKG or ECG) is a test used to check the electrical activity of a patient's heart. An EKG is conducted or performed during routine physical checkups as well as during surgical operations and is a routine and common procedure known in the art.

In surgical pre-operation and post-operation instances, an EKG test may be performed (also known as live EKG monitoring), and may be performed in an Emergency Room (ER), Telemetry floor, Intensive Care Unit (ICU), and Operating Room (OR), for example.

To conduct an EKG, an operator of the EKG is required to connect EKG electrodes to the skin of a patient, which are attached to lead wires or cables that connect to an EKG machine. In the current art, EKG electrodes are attached to a patient's skin via an adhesive element.

Current EKG electrodes that use an adhesive element are disposable and involve an operator peeling soft plastic tabs from a circular or tear shaped element (protecting the adhesive during storage). Upon exposing the adhesive, the operator sticks the EKG electrode directly to the skin of a patient, so that the adhesive element attaches to the patient's skin. However, attachment of an EKG electrode via an adhesive element directly to the skin of a patient has numerous disadvantages, and it is an object of the invention to provide an EKG electrode that overcomes the disadvantages in current EKG electrodes that have this feature.

First off, the adhesive connection in current EKG electrodes often does not stick well to a patient, specifically to the chest of a patient that is hairy, sweaty and/or has moist or oily skin. In such a situation, since the adhesive to skin connection is not good and is not satisfactory for sticking to a patient's skin and maintaining constant contact with the patient's skin during the EKG test, the operator is inconvenienced by having to repeatedly manipulate the adhesive connection to the patient's skin and/or to replace the adhesive connection with a new adhesive or a new EKG electrode.

Another disadvantage of the current EKG electrodes involves the longevity of the integrity of adhesive (aka: the “expiration date” of the adhesive). As current EKG electrodes sit in their packaging, over time the adhesive can expire or becomes less effective. In this situation, if a current EKG electrode is used, the adhesive will expire over time and when the current EKG electrode is applied to a patient, its adhesive strength will already be compromised, so that the adhesive will be weak and the current EKG electrode will not stick well to the skin of a patient.

Another disadvantage involves removal of current EKG electrodes from patients that are hairy and/or have a hairy chest. Upon completion of the EKG test, the current EKG electrodes with adhesive must be removed from a patient's chest. Since the adhesive in current EKG electrodes sticks to a patient's chest and/or to hair on the patient's chest, removal of the EKG from the patient's chest is uncomfortable for the patient as removal of the adhesive causes hair to be pulled and/or removed from the patient's chest which causes pain and discomfort to the patient.

Another disadvantage of current EKG electrodes involves reconnection of the current EKG electrode if/when it becomes disconnected from a lead wire and/or cable. In this instance, the lead wire becomes disconnected from the button on the current EKG electrode, which causes an operator of the EKG to have to reconnect the lead wire to the current EKG electrode to continue the EKG test. Another disadvantage is the reconnection process, which involves force applied to skin and it is often ineffective, requiring the lead to be replaced as it is difficult to attach the wire when the lead electrode is already on the patient.

Another disadvantage of current EKG electrodes involves discarding the current EKG electrodes after they have been used on a patient. Current EKG electrodes with adhesive are discarded after a single use, which is wasteful and bad for the environment, since waste is created after each single use of a current EKG electrode.

Another disadvantage of current EKG electrodes involves patients that are allergic or have hypersensitivity to adhesive and/or to certain materials used with current EKG electrodes. In this instance, use of a current EKG electrode would be contra indicated and/or cannot be used, since allergic patients could develop a rash or other manifestation of allergic reaction from the adhesive element.

Another disadvantage to current EKG electrodes is cost. Since current EKG electrodes are disposed of after each use and millions of EKG tests are conducted each year in the US alone, then millions of current EKG electrodes are disposed of each year. Providing an affordable reusable EKG electrode will reduce costs to hospitals and subsequently to patients and insurance companies.

Another disadvantage to current EKG electrodes is that they use a gel and that at times, the gel may dry up over time or due to poor packaging. Thus, often current EKG electrodes require using additional gel and when necessary masking tape to secure the current EKG electrodes to patients (in the cases in which the adhesive integrity was lost).

Existing EKG electrode systems include U.S. Pat. No. 2,580,628 (Welsh); U.S. Pat. No. 3,640,270 (Hoffmann); U.S. Pat. No. 4,556,065 (Hoffmann); U.S. Pat. No. 4,248,243 (Niess et al.); U.S. Pat. No. 4,137,909 (Hix); U.S. Pat. No. 7,054,677 (Hastings et al.); U.S. Pat. No. 3,534,733 (Phipps); U.S. Pat. No. 4,852,574 (Inoue et al.); U.S. Pat. No. 4,681,118 (Asai et al.); CN204147025U; CN102302364B; CN201542632U; DE2548805A1; EP0289905A1; EP0199694A2; DE3920755C1; and CN201879686U.

However, none of these systems overcomes the above referenced disadvantages in current EKG electrodes. Accordingly, there exists a need to provide an improved EKG electrode that overcomes the problems and disadvantages of current EKG electrodes in the art.

### SUMMARY OF THE INVENTION

To improve upon the prior art, it is an object of the invention to replace and/or supplement the use of current EKG electrodes that are routinely used for any/ALL live, 3-5 lead monitoring EKG electrodes with the apparatus of the present invention. The apparatus of the present invention is designed so as to solve the above referenced disadvantages in current EKG electrodes.

It is an object of the invention to provide a suction cup EKG electrode that uses a refined shape and contour, in conjunction with an ideal material (in terms of function, form, feel) that will create an effective suction to most skin (in terms of elasticity, pliability, emolients, hair) and body shapes/curves/contours.

It is an object of the invention to provide a suction EKG cup electrode that is configured to create an effective suction to the skin of a patient while being comfortable and being able to be applied to and removed from a patient with minimal to zero force and/or discomfort.

It is an object of the invention to provide a suction EKG cup electrode that is configured to be reusable on multiple patients when indicated with cleaning and/or sterilization of the suction cup EKG electrode between uses.

Other objects of the presently claimed invention are directed to solving these issues and to solve existing problems in current EKG electrodes.

These and other objects of the invention are achieved by providing an electrocardiogram apparatus, comprising: a suction cup member, the suction cup member comprising: a suction disc having a concave suction face, a base at a bottom portion of the concave suction face, a convex exterior face and a through-hole in the suction disc, three chambers extending from the concave suction face towards the base at the bottom portion of the concave suction face, wherein compression of at least one of the three chambers creates a negative pressure or a suction force so as to attach the suction cup to the skin of a patient; and an electrode sealed to said suction disc and forming an electrical connection through said through-hole to the skin of the patient.

In certain embodiments, a lead wire is connected to the apex of the suction disc.

In certain embodiments, the three chambers are formed via at least one diaphragm valve that extends from the concave suction face towards the base at the bottom portion of the concave suction face.

In certain embodiments, the suction disc includes at least three diaphragm partitions, such that each of the diaphragm partitions forms a chamber in the suction disc.

In certain embodiments, the diaphragm partitions are constructed from a plastic material, silicone based material or any such deformable material.

In certain embodiments, the suction disc includes a diaphragm or partitions or dividers that form chambers in the suction disc.

In certain embodiments, the suction disc is configured to be compressed via an external force such that upon application of an external force, the volume of at least one of the chambers decreases so as to create a negative pressure or suction force to attach the suction cup to the skin of the patient.

In certain embodiments, the through-hole in the suction disc is located at the center or offset from the center of the suction disc.

In certain embodiments, the suction disc is constructed from an elastic or silicone based material, such that the elastic or silicone based material is configured to be compressed by an external force.

In certain embodiments, the apparatus further comprises at least one relief valve or outlet valve on the convex exterior face of the suction disc, such that the relief valve is configured to release the negative pressure within the chamber in order to remove the suction disc from the skin of a patient. In certain embodiments, the relief valve is configured to release the compressed volume of air so as to generate pressure.

In certain embodiments, there is no valve of any sort within the suction cup.

In certain embodiments, air escapes and renters the suction cup via the edge of the cup in order to effectuate suction or release suction to and from a patient's skin.

In certain embodiments, the electrode comprises: an electrode plate having an inner peripheral edge hermetically sealed to the convex exterior face of said suction disc, and an electrode button extending from the electrode plate, the electrode button configured to receive electrode leads.

In certain embodiments, the electrode plate is located at the top of the convex exterior face of said suction disc.

In certain embodiments, the apparatus further comprises an electrocardiogram gel, the gel configured to be applied within the concave suction face of the suction disc, such that the gel provides an electrical connection from the skin of the patient to the electrode.

In certain embodiments, the electrocardiogram get is located in the vestibule or surrounds the patient end of the electrode.

In certain embodiments, the apparatus further comprises a semi-solid intrinsically conductive polymer (ICP) and/or semi-solid electroconductive polymer in the vestibule of the electrode helping complete the electrical connection to the patient.

In certain embodiments, the ICP and/or semi-solid electroconductive polymer is used with the electrocardiogram gel. In certain embodiments, the ICP and/or semi-solid electroconductive polymer is used as a replacement to the electrocardiogram gel.

In certain embodiments, the ICP and/or semi-solid electroconductive polymer is tacky and sticky and possesses adhesive properties.

In certain embodiments, the semi-solid electroconductive polymer is used to increase electrical conductivity between the suction cup and the patient.

In certain embodiments, the semi-electrocardiogram get is used to increase electrical conductivity between the suction cup and the patient.

In certain embodiments, a portion of the suction cup member is made of an electrically conductive material and a portion of the suction cup member is made of an electrically insulative material.

In certain embodiments, the suction cup member further comprises a channel extending from the electrode to the skin of a patient. In certain embodiments, the electrocardiogram gel is configured to be inserted into the channel such that an electrical connection is formed from the skin of the patient through the gel and to the electrode.

In certain embodiments, the IPC and/or semi-solid electroconductive polymer is located at the end of the channel.

In certain embodiments, the channel is made of electrically conductive material, while the remainder of the suction cup electrode is made of electrically insulative material.

In certain embodiments, there is a gap between the tip of the channel and the skin of a patient.

In certain embodiments, the gap is approximately 1-2 mm in length.

In certain embodiments, the channel is approximately 1-2 cm in diameter.

In certain embodiments, the ICP and/or semi-solid electroconductive polymer or the electrocardiogram gel can be inserted into the gap between the end of the channel and the skin of a patient. In certain embodiments, the ICP and/or semi-solid electroconductive polymer or the electrocardiogram gel is used to provide enhanced conductivity between the skin of the patient and the suction cup electrode.

In certain embodiments, the suction cup includes a check valve within the channel in order to release pressure from the channel into at least one of the chambers.

In certain embodiments, the suction cup includes a spring within the channel. In certain embodiments, the spring is used to control the tension of the suction cup on the skin of a patient.

In certain embodiments, the suction cup includes a check valve body, stainless steel ball within the body, check valve cap and spring.

In certain embodiments, the suction cup includes a central metal connector within the channel.

In certain embodiments, the central metal connector is attached to the channel either via pressure.

In certain embodiments, the central metal connector is attached to the channel by having a flange whose diameter is greater than the channel, holding the central metal connector in place.

In certain embodiments, the suction disc has a semispherical shape or is shaped like a plunger.

In certain embodiments, the suction disc has a protruding surface having a surface gradient suitable to provide suction force.

In certain embodiments, the suction disc has the shape of a triangle.

In certain embodiments, the base of the suction disc provides a seal to the skin of a patient when engaged.

In certain embodiments, the base of the suction disc has an edge/free edge/rim which is flattened and flares out to better complete the seal with the patient's body surface.

In certain embodiments, the base of the bottom portion of the concave suction face attaches to the skin of a patient.

In certain embodiments, the base of the suction disc is textured and/or has a textured material that is configured to interact with the skin of the patient.

In certain embodiments, the suction disc does not use an adhesive material to stick to a surface of a patient. In certain embodiments, the suction disc is devoid of an adhesive material entirely.

In certain embodiments, the suction disc is configured to attach to the skin of a patient using only a suction force or a negative pressure.

In certain embodiments, the suction cup member includes one or more ridges on the convex exterior face. In certain embodiments, the suction cup member includes two ridges on the convex exterior face. In certain embodiments, the suction cup member includes three ridges on the convex exterior face. In certain embodiments, the suction cup member includes four ridges on the convex exterior face. In certain embodiments, the suction cup member includes five ridges on the convex exterior face.

In certain embodiments, each of the one or more ridges are configured to a human finger or thumb, such that actuation of each of the ridges causes the chambers to compress and the suction force to be applied.

In certain embodiments, the ridges allow for direct compression of the chambers.

In certain embodiments, the suction cup member includes flanges that are external to the suction cup member. In certain embodiments, the suction cup member includes demarcated areas on the suction cup member, such that the demarcated areas correspond to a human finger or thumb, such that actuation of each of the demarcated areas causes the chambers to compress and the suction force to be applied.

In certain embodiments, the suction cup member has a domed shape that ensures that there are no corners or crevices that would preclude quick effective suction or effective, easy cleaning/disinfection/sterilization after use.

Other objects of the invention are achieved by providing a system for recording an electrocardiogram, the system comprising: at least one suction cup member, the suction cup member comprising: a suction disc having a concave suction face, a base at a bottom portion of the concave suction face, a convex exterior face and a through-hole in the suction disc, one or more chambers extending from the concave suction face towards the base at the bottom portion of the concave suction face, wherein compression of at least one of the chambers creates a negative pressure or a suction force so as to attach the suction cup to the skin of a patient; an electrode hermetically sealed to said suction disc and forming an electrical connection through said through-hole to the patient; one or more lead wires extending from said electrode and connecting said at least one electrode to an electrocardiogram or transducer machine.

In certain embodiments, the system includes three chambers extending from the concave suction face towards the base at the bottom portion of the concave suction face.

In certain embodiments, the system includes two chambers extending from the concave suction face towards the base at the bottom portion of the concave suction face.

In certain embodiments, the system includes one chamber extending from the concave suction face towards the base at the bottom portion of the concave suction face.

Other objects of the invention are achieved by providing an electrocardiogram apparatus, comprising: a suction cup member, the suction cup member comprising: a suction disc having a concave suction face, a base at a bottom portion of the concave suction face, a convex exterior face and a through-hole in the suction disc, at least two chambers extending from the concave suction face towards the base at the bottom portion of the concave suction face, wherein compression of at least one of the at least two chambers creates a negative pressure or a suction force so as to attach the suction cup to the skin of a patient; and an electrode sealed to said suction disc and forming an electrical connection through said through-hole to the skin of the patient.

Other objects of the invention are achieved by providing an electrocardiogram apparatus, comprising: a suction cup member, the suction cup member comprising: a suction disc having a concave suction face, a base at a bottom portion of the concave suction face, a convex exterior face and a through-hole in the suction disc, one chamber extending from the concave suction face towards the base at the bottom portion of the concave suction face, wherein compression of the one chamber creates a negative pressure or a suction force so as to attach the suction cup to the skin of a patient; and an electrode sealed to said suction disc and forming an electrical connection through said through-hole to the skin of the patient.

In certain embodiments, the suction disc includes no diaphragm partitions, such that the suction disc has a single chamber.

In certain embodiments, there is no valve of any sort. In certain embodiments, air escapes and renters the suction cup via the edge of the cup.

Other objects of the invention are provided by a method of using the electrocardiogram apparatus and suction cup as set forth above.

Other objects of the invention and its particular features and advantages will become more apparent from consideration of the following drawings and accompanying detailed description. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

FIG. 1 is a top perspective view of an EKG suction cup electrode of an embodiment of the present invention;

FIG. 2 is a bottom perspective view of the EKG suction cup electrode of FIG. 1;

FIG. 3A is a side view of the EKG suction cup electrode of FIG. 1 in one embodiment;

FIG. 3B is a side view of the EKG suction cup electrode of FIG. 1 in another embodiment;

FIG. 4A is an exterior view of a valve of the EKG suction cup electrode of FIG. 1;

FIG. 4B is an exterior view of a valve of the EKG suction cup electrode of FIG. 1;

FIG. 5 is a bottom view of an EKG suction cup electrode of an embodiment of the present invention; and

FIG. 6 is a bottom view of an EKG suction cup electrode of an embodiment of the present invention.

### DETAILED DESCRIPTION OF THE INVENTION

In the following description, numerous details are set forth for purpose of explanation. However, one of ordinary skill in the art will realize that the invention may be practiced without the use of these specific details. For instance, the techniques described below are described in a specified order, but other embodiments may change the order of the operations while still embodying the current invention.

Moreover, certain features shown in certain embodiments and figures are contemplated to be included in other embodiments, even if not expressly shown in the features in certain embodiments.

As set forth above, the invention is directed to a suction cup EKG electrode. Suction cups are commonly made of rubber molded material. In applying a suction cup to a pane or sheet of glass or other material the concave suction face is placed against the glass, and then the suction cup is pressed toward the glass to flatten the concave face and expel the air from between the concave face and the glass. However, for use in patients, suction cup EKG electrodes have special challenges that are different to that of simply putting a suction cup against glass.

To place a suction cup EKG electrode on a patient, an operator would perform the following: (1) ensure a clean skin surface as a contact site; (2) place a small amount of gel on either/and/or/both: the patient, and the suction cup (i.e. within the concave portion of the suction cup or within the patient end of the vestibule of the channel housing the electrode); and (3) with minimal pressure on the patient's skin surface, the suction cup will usually stick with more than sufficient negative pressure.

To remove the suction cup, the operator would use minimal force by; simply and easily changing the conformation of the device (such as by turning and/or rotating the suction cup), which, in turn, will quickly disrupt the ability of the device to maintain its negative pressure grip.

The operator would then wipe off all sites of gel on both patient and EKG electrodes and the suction cup and the electrode unit can easily be disinfected with special alcohol dispenser or any alcohol source for reuse.

In certain embodiments, the operator may perform additional steps of the method.

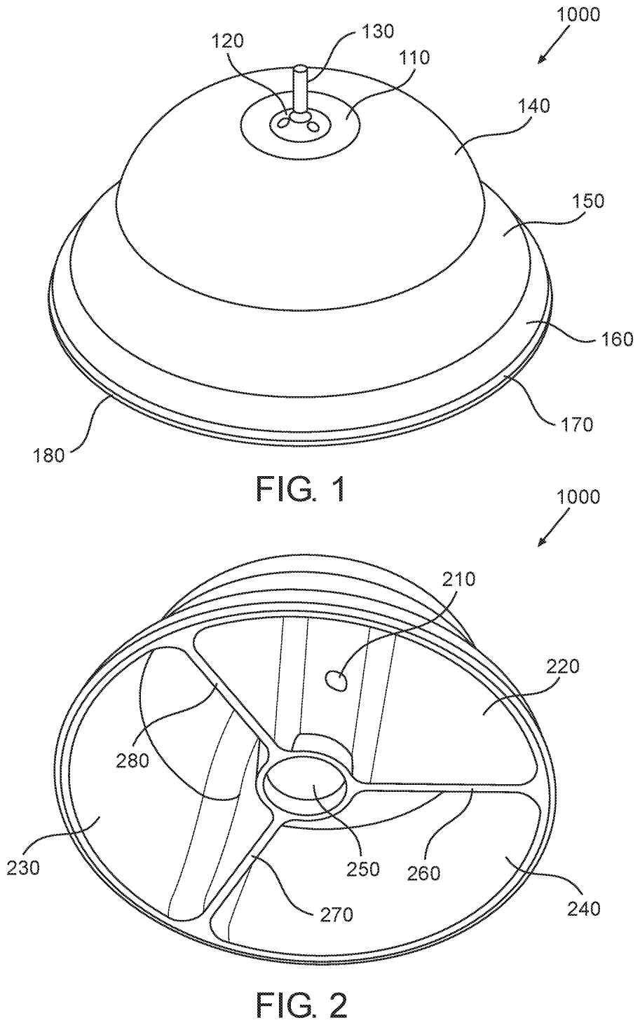

FIG. 1 is directed to an EKG suction cup electrode of an embodiment of the present invention. FIG. 1 shows the suction cup member 1000 having a suction cup exterior 140/150/160/170 and electrode 110 sealed to said suction cup exterior 140/150/160/170. The suction cup exterior 140/150/160/170 is shown being made of multiple sections, however, one or more sections are contemplated to form the suction cup exterior. In certain embodiments, the one or more section can be fused as a single section. The suction cup member 1000 is also shown having a lip or base 180, which is used to interface with the skin of a patient. Also, the electrode 110 is connected to the top and central portion of the suction cup member. In certain embodiments, the electrode is fused to the suction cup exterior 140/150/160/170 and in other embodiments, it rests within a plate located on top of the suction cup member.

In certain embodiments, the electrode 110 forms a connection button type connection with the suction cup exterior or suction cup housing.

Also shown in FIG. 1, is an electrode lead wire 130 and a relief valve exit 120. The relief valve (also known as an outlet valve) is used to displace air within the suction cup electrode when a user presses the exterior of the suction cup electrode to deform it. The electrode lead wire 130 is connected to the EKG machine.

In certain embodiments, relief valve is a one-way valve to expel air and configuration of the valve allows for generation of negative pressure in the concave area of the suction cup.

In certain embodiments, the relief valve is made of an electrically conductive material. In certain embodiments, the relief valve is connected to an electrode.

In certain embodiments, the relief valve includes one or more exits 120, such that the one or more exits allow for increased airflow through the relief valve and out of the suction cup.

In certain embodiments, the electrode lead wire 130 can be permanently attached to the suction disc and in other embodiments, the electrode lead wire 130 is able to be attached and detached from the suction disc.

In certain embodiments, the cup exterior 140/150/160/170 has a convex exterior shape.

FIG. 2 shows a bottom perspective view the EKG suction cup electrode of FIG. 1. Here the three chambers 220, 230 and 240 are shown. Furthermore, channel 250 is shown as well as exit hole 210. Exit hole 210 can be a bore and/or and directed the flow of air in one direction

FIG. 2 shows diaphragms 260, 270 and 280 separating the three chambers 220, 230 and 240.

In certain embodiments, the chambers 220, 230 and 240 are shown such that compression of at least one of the three chambers creates a negative pressure or a suction force so as to attach the suction cup to the skin of a patient.

In certain embodiments, exit hole 210 is either provided or not required by the suction cup 1000. In certain embodiments, the exit hole 210 is sealed such that there is no exit from the channel 250 to any of the three chambers 220, 230 and 240. In such an embodiment, there is contemplated to be no internal valve or assembly within the suction cup. In such an embodiment, air escapes and re-enters the chambers via the edge of the suction cup.

In certain embodiments, the channel 250 is made of conductive material, such as metal, while the three chambers 220, 230 and 240 are made of an insulative material such as plastic or a polymer.

In certain embodiments, the channel is cylindrical and has a metal conductive portion embedded in the wall of the channel.

In certain embodiments, the channel is made up of an electrically conductive polymer material.

In certain embodiments, the channel includes a central metal connector located within the channel.

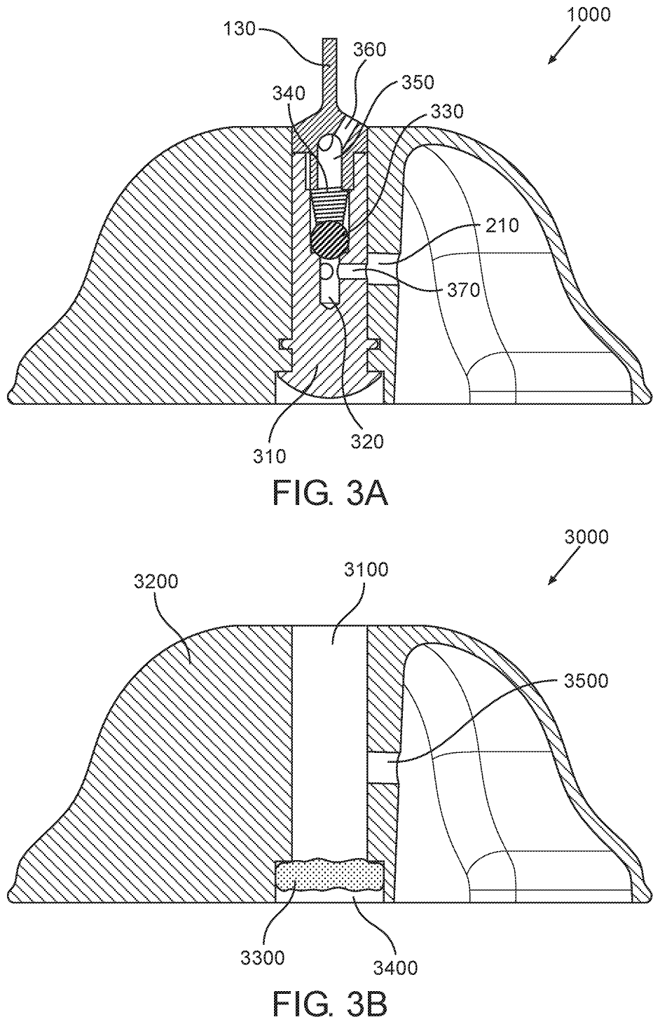

FIG.3A is a side view of the EKG suction cup electrode of FIG. 1 in one embodiment. Here, the suction cup is shown having electrode lead 130 and valve 310. The valve includes an upper cap portion 350, spring 340, O-ring 330, bottom portion 320, exit 370 and relief valve 360. The valve is fitted within channel 250. The channel has an exit hole (bore) 210 that allows pressure from the valve to be released into one or more of the three chambers 220, 230 and 240. In certain embodiments, multiple exit holes (bores) are contemplated in the channel.

FIG. 3B shows an alternative embodiment of the invention without the valve assembly. FIG. 3B shows a suction cup 3000 having an exterior cup surface 3200, channel 3100 and bore 3500.

In certain embodiments, the channel is able to conduct current and is made of an electrically conductive material, while the remainder of the suction cup is made of electrically insulative material.

In certain embodiments, bore 3500 allows for pressure or electrical current to be passed from channel 3100. However, in other embodiments, bore 3500 is not present and is not essential. In such embodiments, the channel is sealed from the reminder of the one or more of the three chambers 220, 230 and 240. In such an embodiment, air escapes and re-enters the chambers via the edge of the suction cup, once one of the chambers are deformed or manipulated by a user.

In certain embodiments, the edge/free edge/rim of the cup is flattened and flares out to better complete the seal with the patient's skin.

The suction cup exterior of FIGS. 1-3B can be attached to the skin of the patient via the bottom lip portion 180. As shown in FIG. 3B, a gel and/or a polymer 3300 can be inserted into the channel.

In certain embodiments, there is a gap 3400 formed at the bottom of the channel such that the gel and/or a polymer 3300 can be inserted into the gap 3400 to provide electrical conductivity from the channel to the skin of the patient.

This is especially useful as the channel can be made of metal or other rigid materials that would not be comfortable to have touch the skin of a patient. The gel and/or a polymer 3400 provide the ability for user comfort as these elements are configured to touch the skin of a patient rather than the channel.

In certain embodiments, the suction cup has a barrier between the channel and the chambers, such that the gel and/or a polymer 3300 are prevented from entering the chambers.

Furthermore, it is contemplated that the channel is shorter than maximum height of the suction cup electrode in order to produce a gap between the distal end of the channel and the skin of a patient.

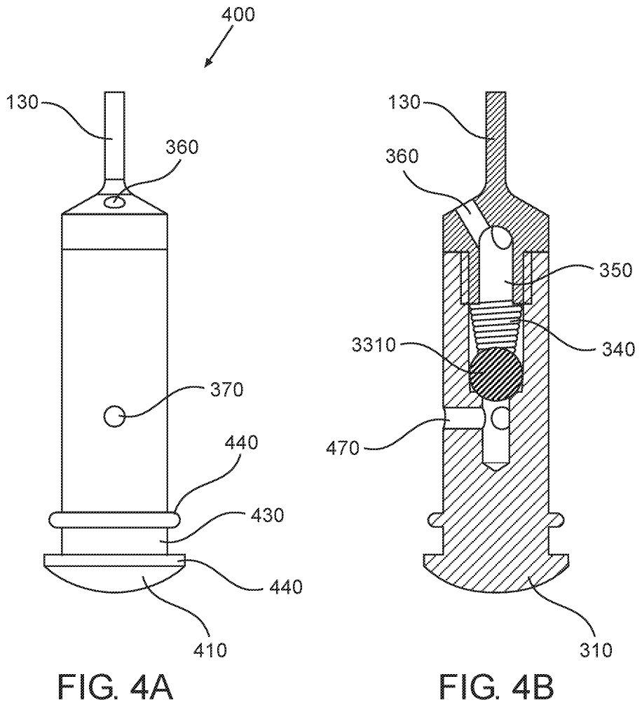

FIGS. 4A-4B is an exterior and interior view of a valve 310 of the EKG suction cup electrode 1000. The valve includes an upper cap portion 350, spring 340, O-ring 330, bottom portion 320, exit 370 and relief valve 360. The valve has one or more flanges 440, a central housing 430 and a bottom housing 440 having a bottom end cap 410. The valve is configured to fit within channel 250 such that the one or more flanges 440 are accepted by the internal walls of the channel 250 and exert a pressure on the channel such that the valve is pressure-fit within the channel.

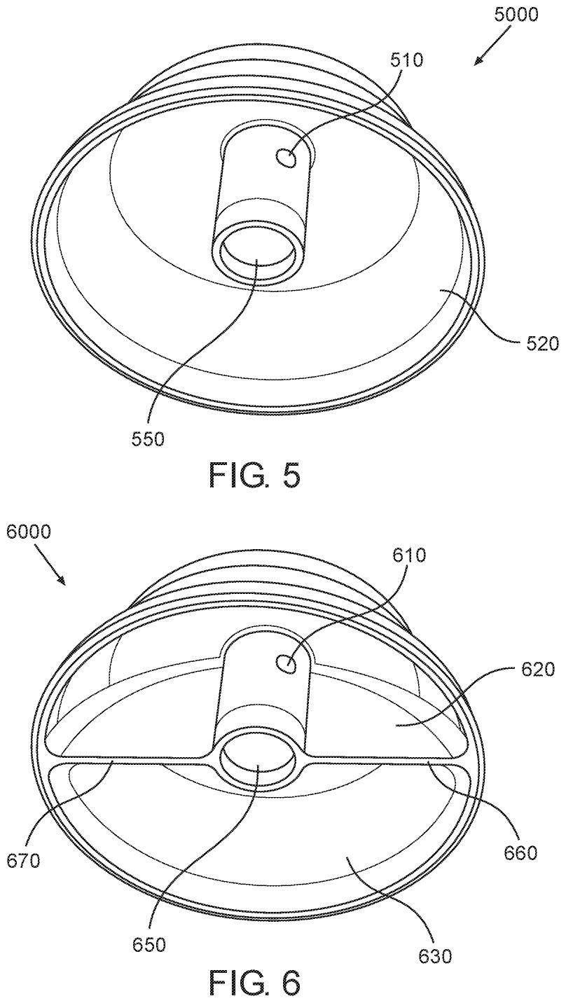

FIG. 5 is a bottom view of an EKG suction cup electrode 5000 of an alternate embodiment of the present invention. In FIG. 5, channel 550 is shown as well as bore 510. The suction cup housing 520 is shown. FIG. 5 is a similar embodiment to that of FIGS. 1-4B such that FIG. 5 is configured to include a valve an operate in a similar manner to that of FIG. 1. However, FIG. 5 has a single chamber, rather than three chambers shown in FIG. 1.

FIG. 6 is a bottom view of an EKG suction cup electrode 6000 of an alternate embodiment of the present invention. In FIG. 6, channel 650 is shown as well as bore 610. The suction cup housing has two chambers 620 and 630, which are separated by diaphragms 660 and 670. FIG. 5 is a similar embodiment to that of FIGS. 1-4B such that FIG. 6 is configured to include a valve an operate in a similar manner to that of FIG. 1. However, FIG. 6 has two chambers, rather than three chambers shown in FIG. 1.

As contemplated by the invention the single, dual and tri-chambered suction cup has a concave shape with a base portion.

In certain embodiments, the base portion is made of thicker material than the rest of the suction cup. In certain embodiments, the base portion is textured so as to easily grip the skin of a patient.

In certain embodiments, the exterior housing of the suction cup has flanges and other grip holds so as to make it easy to grasp the exterior of the suction cup.

In certain embodiments, the exterior housing has a structural support member to keep the housing in place such that only a portion of the exterior housing is deformable, while the other portion is not deformable and able to keep its shape.

In operation, the exterior of the suction cup electrode is flexible and is deformable. It can be placed upon the skin of a user by pressing upon the housing to deform it and to release pressure within the one or more chambers.

To place the suction cup on the skin of a patient, a similar operation persists. Here, suction cup is placed on the skin of the user and the housing is pressed so it is deformed. Upon release of exterior of the housing, the suction cup reverts to its uncompressed position and pressure within the suction cup is released, thus causing the suction cup to “suction” to the skin of a patient.

In certain embodiments of the invention, the one or more chambers are each configured to be individually compressed and to create suction against the skin of a patient.

In certain embodiments, the outlet vales are provided (not shown) on the exterior housing of the chambers. In certain embodiments, outlet valves on each of the three chambers are configured to be one-way valves and can release air from inside the chamber in order to create a negative pressure and suction to a patient's skin.

In certain embodiments, the suction disc includes at least one demarcated area, ridge or flange, such that an operator's fingers and thumbs can contact the at least one demarcated area, ridge or flange in order to compress the at least one chamber in the suction cup so as to compress the suction cup to create a negative pressure and to attach to a patient.

While the invention has been specifically described in connection with certain specific embodiments thereof, it is to be understood that this is by way of illustration and not of limitation and that various changes and modifications in form and details may be made thereto, and the scope of the appended claims should be construed as broadly as the prior art will permit.

The description of the invention is merely exemplary in nature, and thus, variations that do not depart from the gist of the invention are intended to be within the scope of the invention. Such variations are not to be regarded as a departure from the spirit and scope of the invention.

1. An electrocardiogram apparatus, comprising:
a suction cup member, the suction cup member comprising:
a suction disc having a concave suction face, a base at a bottom portion of the concave suction face, a convex exterior face and a through-hole in the suction disc,
three chambers extending from the concave suction face towards the base at the bottom portion of the concave suction face,
wherein compression of at least one of the three chambers creates a negative pressure or a suction force so as to attach the suction cup to the skin of a patient; and
an electrode sealed to said suction disc and forming an electrical connection through said through-hole to the skin of the patient.

2. The electrocardiogram apparatus of claim 1, wherein the three chambers are formed via at least one diaphragm partition that extends from the concave suction face towards the base at the bottom portion of the concave suction face.

3. The electrocardiogram apparatus of claim 2, wherein the suction disc includes at least three diaphragm partitions, such that each of the diaphragm partitions forms a chamber in the suction disc.

4. The electrocardiogram apparatus of claim 2, wherein the diaphragm are constructed from a plastic material, silicone based material or any such deformable material.

5. The electrocardiogram apparatus of claim 1, the suction disc is configured to be compressed via an external force such that upon application of an external force, the volume of at least one of the chambers decreases so as to create a negative pressure or suction force sufficient to attach the suction cup to the skin of the patient.

6. The electrocardiogram apparatus of claim 1, wherein the through-hole in the suction disc is located at the center or offset from the center of the suction disc.

7. The electrocardiogram apparatus of claim 1, wherein the suction disc is constructed from an elastic or silicone based material, such that the elastic or silicone based material is configured to be compressed by an external force.

8. The electrocardiogram apparatus of claim 1, further comprising at least one relief valve on the convex exterior face of the suction disc, such that the relief valve is configured to release the negative pressure within the chamber in order to remove the suction disc from the skin of a patient.

9. The electrocardiogram apparatus of claim 1, wherein the electrode comprises:
an electrode plate having an inner peripheral edge hermetically sealed to the convex exterior face of said suction disc, and
an electrode button extending from the electrode plate, the electrode button configured to receive electrode wires from a monitoring device.

10. The electrocardiogram apparatus of claim 1, further comprising an electrocardiogram gel, the gel configured to be applied within a patient end of the concave suction face of the suction disc, such that the gel provides an electrical connection from the skin of the patient to the electrode, wherein a portion of cup member is made of an electrically conductive material and a portion of the cup member is made of an electrically insulative material.

11. (canceled)

12. (canceled)

13. The electrocardiogram apparatus of claim 1, wherein the suction cup member further comprises a channel extending from the electrode to the skin of a patient.

14. (canceled)

15. (canceled)

16. The electrocardiogram apparatus of claim 13, wherein, the channel is made of electrically conductive material, while the remainder of the suction cup electrode is made of electrically insulative material.

17. The electrocardiogram apparatus of claim 1, wherein the suction disc has a semispherical shape or is shaped like a plunger.

18. The electrocardiogram apparatus of claim 1, wherein the suction disc has a protruding surface having a surface gradient suitable to provide suction force, wherein the suction disc does not use an adhesive material to stick to a surface of a patient.

19. (canceled)

20. (canceled)

21. The electrocardiogram apparatus of claim 1, wherein the electrocardiogram electrode is configured to attach to the skin of a patient using only a suction force or a negative pressure.

22. (canceled)

23. (canceled)

24. (canceled)

25. (canceled)

26. The electrocardiogram apparatus of claim 21, wherein the suction cup member includes two ridges on the convex exterior face.

27. The electrocardiogram apparatus of claim 26, wherein each of the two ridges are configured to a human finger or thumb, such that actuation of each of the ridges causes the chambers to compress and the suction force to be applied.

28. A system for recording an electrocardiogram, the system comprising:
at least one suction cup member, the suction cup member comprising:
a suction disc having a concave suction face, a base at a bottom portion of the concave suction face, a convex exterior face and a through-hole in the suction disc,
one or more chambers extending from the concave suction face towards the base at the bottom portion of the concave suction face,
wherein compression of at least one of the chambers creates a negative pressure or a suction force so as to attach the suction cup to the skin of a patient;
an electrode hermetically sealed to said suction disc and forming an electrical connection through said through-hole to the patient; and
one or more lead wires extending from said electrode and connecting said at least one electrode to an electrocardiogram or transducer machine.

29. The system of claim 28, wherein the system includes three chambers extending from the concave suction face towards the base at the bottom portion of the concave suction face.

30. An electrocardiogram apparatus, comprising:
a suction cup member, the suction cup member comprising:
a suction disc having a concave suction face, a base at a bottom portion of the concave suction face, a convex exterior face and a through-hole in the suction disc,
at least two chambers extending from the concave suction face towards the base at the bottom portion of the concave suction face,
wherein compression of at least one of the at least two chambers creates a negative pressure or a suction force so as to attach the suction cup to the skin of a patient; and
an electrode sealed to said suction disc and forming an electrical connection through said through-hole to the skin of the patient.

31. (canceled)

32. (canceled)
